(19) United States
(12) Patent Application Publication
(10) Pub. No.: US 2025/0014753 A1
(43) Pub. Date: Jan. 9, 2025

(54) DISEASE RISK ANALYSIS APPARATUS, DISEASE RISK ANALYSIS METHOD, AND STORAGE MEDIUM

(71) Applicant: KABUSHIKI KAISHA TOSHIBA, Tokyo (JP)

(72) Inventor: Masahiro OZAWA, Yokohama Kanagawa (JP)

(73) Assignee: KABUSHIKI KAISHA TOSHIBA, Tokyo (JP)

(21) Appl. No.: 18/589,623

(22) Filed: Feb. 28, 2024

(30) Foreign Application Priority Data
Jul. 6, 2023 (JP) 2023-111488

(51) Int. Cl. G16H 50/30; G16H 50/70

(52) U.S. Cl. CPC G16H 50/30; G16H 50/70

(57) ABSTRACT

According to one embodiment, a disease risk analysis apparatus includes a processor. The processor acquires healthcare data including genetic score data and temporal data collected over time. The processor determines a threshold for stratifying a genetic score. The processor stratifies the genetic score data based on the threshold. The processor sets a criterion for at least a test value of the temporal data. The processor generates first observation target data from the temporal data. The processor generates starting point data based on the first observation target data and the criterion. The processor determines an observation target from the temporal data. The processor generates second observation target data.

### CROSS-REFERENCE TO RELATED APPLICATIONS

This application is based upon and claims the benefit of priority from the Japanese Patent Application No. 2023-111488, filed Jul. 6, 2023, the entire contents of which are incorporated herein by reference.

### FIELD

Embodiments described herein relate generally to a disease risk analysis apparatus, a disease risk analysis method, and a storage medium.

### BACKGROUND

In recent years, it has become possible to estimate genetic differences for various diseases and diatheses by a polygenic risk scores (PRS) calculated from individual genome data. The PRS is a score representing a genetic risk for various diseases and diatheses in which a large number of genes are involved in the onset.

According to the PRS, for example, various analyses can be performed, such as correlation analysis, by using cross-sectional data, between the PRS and the presence or absence of a medical history, and estimation of a disease risk at a certain age in a case where temporal data is stratified by the PRS.

In order to reduce future diseases, it is important to implement appropriate preventive measures if the future disease risk is predicted to be high. For prediction of a future disease risk, it is conceivable to use temporal data obtained by collecting health checkup results, medical examination results, and the like over time. Some people have a low risk of disease onset and other people have a high risk of disease onset due to genetic differences even if they have the same test values in the health checkup results. In addition, some people have a fast period from the onset of a disease to the development of complications and other people have a slow period from the onset of a disease to the development of complications due to genetic differences even if they were found to develop the disease at the same time in the medical examination. Such temporal data may not be appropriately analyzed with simple PRS stratification.

### BRIEF DESCRIPTION OF THE DRAWINGS

FIG. 1 is a diagram illustrating a configuration of an example of a disease risk analysis apparatus according to an embodiment.

FIG. 2 is a diagram illustrating an example of PRS data.

FIG. 3 is a diagram illustrating an example of health checkup data.

FIG. 4 is an example of medical examination data.

FIG. 5 is a diagram illustrating an example of a hardware configuration of the disease risk analysis apparatus.

FIG. 6 is a flowchart illustrating operations of the disease risk analysis apparatus.

FIG. 7 illustrates stratified data that is stratified by diabetes PRS threshold.

FIG. 8 is a diagram illustrating an example of first observation target data.

FIG. 9 is a diagram illustrating an example of starting point data.

FIG. 10 is a diagram illustrating an example of second observation target data.

FIG. 11A is a graph obtained by selecting HbA1c as the second observation target data and stratifying HbA1c by the PRS of BMI.

FIG. 11B is a graph obtained by selecting the highest blood pressure as the second observation target data and stratifying the highest blood pressure by the PRS of BMI.

FIG. 11C is a graph obtained by selecting LDL as the second observation target data and stratifying LDL by the PRS of BMI.

FIG. 11D is a graph obtained by selecting HDL as the second observation target data and stratifying HDL by the PRS of BMI.

FIG. 11E is a graph obtained by selecting TG as the second observation target data and stratifying TG by the PRS of BMI.

FIG. 11F is a graph obtained by selecting BMI as the second observation target data and stratifying BMI by the PRS of BMI.

FIG. 11G is a graph obtained by selecting the body weight as the second observation target data and stratifying the body weight by the PRS of BMI.

FIG. 11H is a graph obtained by selecting GOT as the second observation target data and stratifying GOT by the PRS of BMI.

FIG. 11I is a graph obtained by selecting GPT as the second observation target data and stratifying GPT by the PRS of BMI.

FIG. 11J is a graph obtained by selecting γGTP as the second observation target data and stratifying γGTP by the PRS of BMI.

FIG. 12A is a graph obtained by selecting the prevalence of diabetes as the second observation target data and stratifying the prevalence of diabetes with the PRS of the highest blood pressure.

FIG. 12B is a graph obtained by selecting the prevalence of hypertension as the second observation target data and stratifying the prevalence of hypertension by the PRS of the highest blood pressure.

FIG. 12C is a graph obtained by selecting the prevalence of dyslipidemia as the second observation target data and stratifying the prevalence of dyslipidemia by the PRS of the highest blood pressure.

FIG. 12D is a graph obtained by selecting the prevalence of obesity as the second observation target data and stratifying the prevalence of obesity by the PRS of the highest blood pressure.

FIG. 12E is a graph obtained by selecting the prevalence of liver dysfunction as the second observation target data and stratifying the prevalence of liver dysfunction by the PRS of the highest blood pressure.

### DETAILED DESCRIPTION

In general, according to one embodiment, a disease risk analysis apparatus includes a processor including hardware. The processor acquires healthcare data including genetic score data holding a genetic score for each user and temporal data including at least one of health checkup data and medical examination data for each user collected over time. The processor determines a threshold for stratifying the genetic score. The processor stratifies the genetic score data based on the threshold to generate stratified data. The processor sets a criterion for at least a test value of the health checkup data and/or at least a medical examination status of the medical examination data. The processor generates first observation target data by extracting a test value and/or a medical examination status corresponding to the criterion from the temporal data. The processor generates starting point data based on the first observation target data and the criterion. The processor determines an observation target from at least a test value of the health checkup data and/or at least a medical examination status of the medical examination data. The processor generates second observation target data by extracting the test value and/or the medical examination status determined as the observation target with the starting point data as a starting point from the temporal data. The processor analyzes the second observation target data by stratifying the second observation target data based on the stratified data.

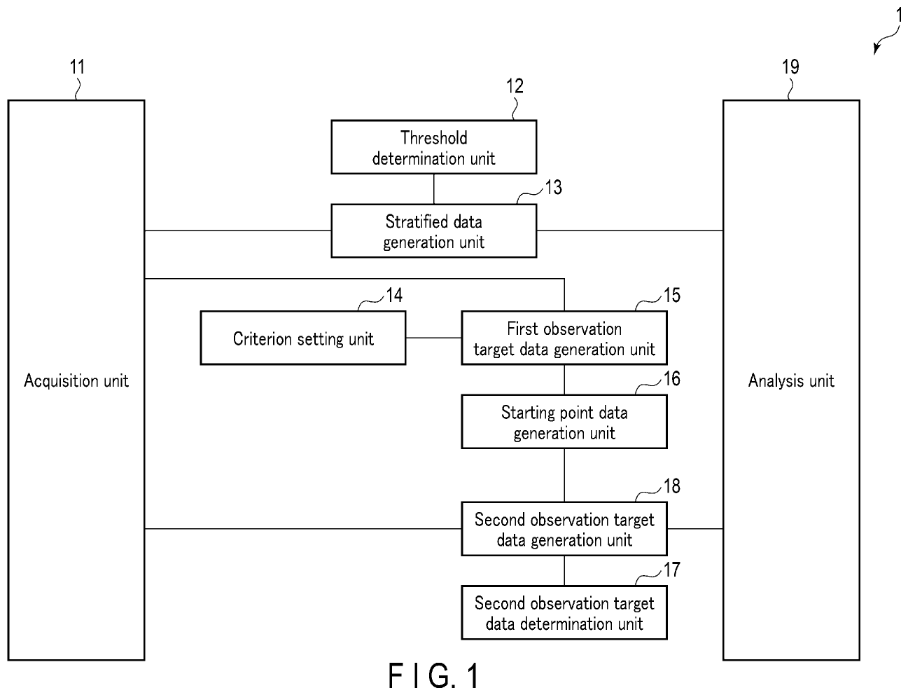

Hereinafter, embodiments will be described with reference to the drawings. FIG. 1 is a diagram illustrating a configuration of an example of a disease risk analysis apparatus according to an embodiment. The disease risk analysis apparatus 1 includes an acquisition unit 11, a threshold determination unit 12, a stratified data generation unit 13, a criterion setting unit 14, a first observation target data generation unit 15, a starting point data generation unit 16, a second observation target data determination unit 17, a second observation target data generation unit 18, and an analysis unit 19.

The acquisition unit 11 acquires healthcare data. The healthcare data is personal data of each user related to prediction of the disease risk of the user. The healthcare data includes PRS data. The healthcare data also includes temporal data. The healthcare data can be input by any method such as operation of the disease risk analysis apparatus 1 by a disease risk analyst such as a doctor.

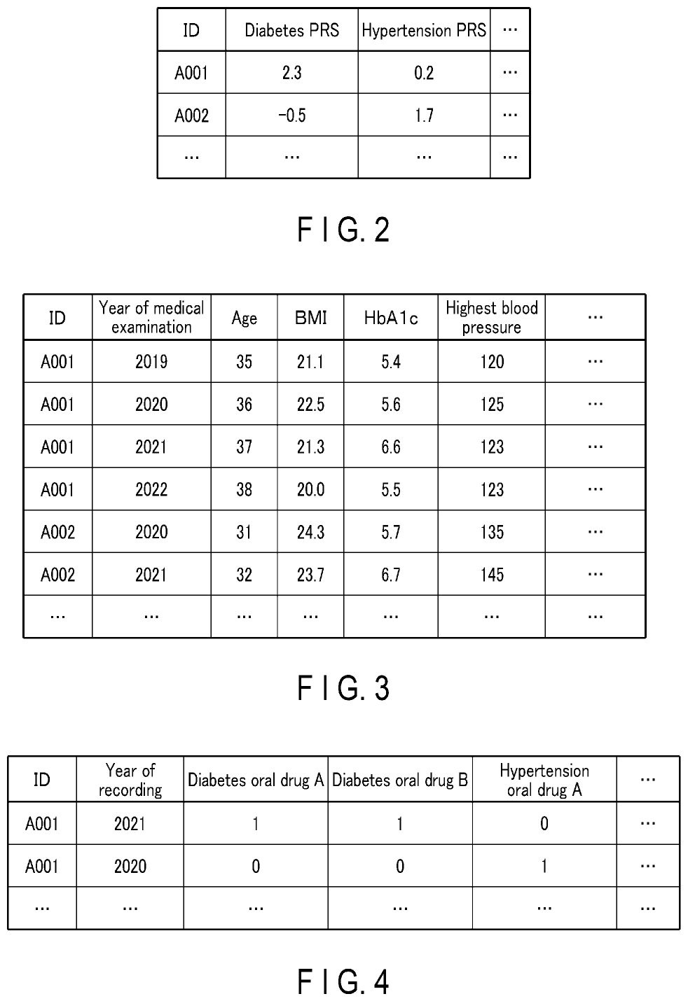

The PRS data is data that holds a value of PRS as a genetic score for each user that is a disease risk analysis target. FIG. 2 is a diagram illustrating an example of PRS data. As illustrated in FIG. 2, the PRS data has IDs and values of PRS. The ID is a character string uniquely assigned to each user. The PRS is a score representing a genetic difference for various diseases and diatheses in which a large number of genes are involved in the onset. The PRS data is associated with each disease and diathesis. FIG. 2 provides diabetes PRS and hypertension PRS as examples. Diabetes PRS is a score representing genetic susceptibility to diabetes. On the other hand, hypertension PRS is a score representing genetic susceptibility to hypertension. The PRS in the embodiment may be PRS having a correlation with the risk of onset of obesity, hypertension, diabetes, dyslipidemia, and liver dysfunction, and PRS having a correlation with body mass index (BMI), hemoglobin A1c (HbA1c), highest blood pressure, low density lipoprotein (LDL), high density lipoprotein (HDL), triglyceride (TG), body weight, glutamic oxaloacetate transaminase (GOT), glutamic pyruvic transaminase (GPT), and γGTP as test values.

The temporal data includes at least one of health checkup data and medical examination data for each user that are collected over time. The health checkup data is data of the results of health checkup of each user. The medical examination data is data of medical examination status of each user in a medical institution.

FIG. 3 is a diagram illustrating an example of health checkup data. The health checkup data can be generated from the results of a health checkup conducted every year for each user. As illustrated in FIG. 3, the health checkup data includes ID, year of health checkup, age, and test values. The ID is a character string uniquely assigned to each user. The ID in the PRS data and the ID in the health checkup data are common. The year of health checkup is the year in which the user with the corresponding ID has received the health checkup. The age is the age of the user with the corresponding ID at the time of receiving the health check. The test values are various test values that can be used as criteria for determining the onset of obesity, hypertension, diabetes, dyslipidemia, and liver dysfunction. The health checkup data herein may include information other than the test values. For example, the health checkup data may include information obtained by an inquiry of a medical history or the like.

FIG. 4 is an example of medical examination data. The medical examination data is generated from information included in health insurance claims, for example. The health insurance claims include information on various medical examination status such as disease names, medical actions, prescribed medicines, and the like for each user who received a medical examination in a medical institution. The medical examination data in FIG. 4 is data including a medical examination status indicating whether the user with each ID received a prescription of a medicine related to a disease in each year. The medical examination status indicates the status of various medical examinations that can serve as criteria for determining the onset of obesity, hypertension, diabetes, dyslipidemia, and liver dysfunction. In FIG. 4, “1” is recorded if a medicine prescription was received, and “0” is recorded if no medicine prescription was received. The medicine may be a specific medicine or a combination of a plurality of medicines. The medical examination data may be data further including a medical examination status as to whether the user received a specific medical action.

The threshold determination unit 12 determines the PRS used for stratification of the temporal data and the threshold of the PRS used for stratification. The PRS used for stratification and its threshold can be determined in response to an operation of the disease risk analysis apparatus 1 by a disease risk analyst such as a doctor, for example. The threshold is determined so as to divide the users into top 10%, middle 80%, and bottom 10%, for example. The present invention is not limited thereto, and the threshold may be arbitrarily determined so as to divide the users into top 33%, middle 34%, and bottom 33%, for example. Furthermore, the threshold is not necessarily set so as to divide the users into three groups, and may be set so as to divide the users into two groups, or may be set so as to divide the users into four or more groups. In addition, the threshold may be set for a plurality of PRSs.

Furthermore, the threshold may be a fixed value or a variable value set by operation of the disease risk analysis apparatus 1 by a disease risk analyst such as a doctor.

The stratified data generation unit 13 generates stratified data by stratifying the PRS data based on the threshold of the PRS determined by the threshold determination unit 12. The stratified data is data in which a label representing each layer is associated with each ID of the PRS data stratified by the threshold.

The criterion setting unit 14 sets a criterion for the first observation target data for determining the starting point of the change in the temporal data. The first observation target data is selected from at least one test value of the health checkup data and/or at least one medical examination status of the medical examination data. The first observation target data and the criterion therefor can be set by an arbitrary method such as operation of the disease risk analysis apparatus 1 by a disease risk analyst such as a doctor.

The first observation target data generation unit 15 generates the first observation target data from the temporal data based on the criterion set by the criterion setting unit 14. The first observation target data is generated by extracting the test value and/or the medical examination status corresponding to the criterion from the health checkup data and/or the medical examination data. In a case where a plurality of criteria is set, the first observation target data for the test value and/or the medical examination status corresponding to each criterion can be generated.

The starting point data generation unit 16 generates starting point data based on the first observation target data. The starting point data is data in which a year of medical examination in which the test value with a certain ID of the first observation target data reached the criterion is set as the 0th year as a starting point, and other years of medical examination with the same ID are held as relative years from the 0th year. The starting point data may be data that holds only the relative years earlier than the 0th year, may be data that holds only the relative years later than the 0th year, or may be data that holds the relative years earlier and later than the 0th year. The starting point data may be data that holds only a plurality of relative years closest to the 0th year. The setting of the data held as the starting point data can be set by an arbitrary method such as setting in response to an operation of the disease risk analysis apparatus 1 by a disease risk analyst such as a doctor.

The second observation target data determination unit 17 determines the second observation target data in the temporal data. The second observation target data is determined from at least one test value of the health checkup data and/or at least one medical examination status of the medical examination data. The second observation target data can be selected by an arbitrary method such as setting in response to an operation of the disease risk analysis apparatus 1 by a disease risk analyst such as a doctor.

The second observation target data generation unit 18 generates the second observation target data based on the temporal data acquired by the acquisition unit 11, the starting point data generated by the starting point data generation unit 16, and the result determined by the second observation target data determination unit 17. The second observation target data is data obtained by extracting the test value and/or the medical examination status determined as the second observation target data in the temporal data with the relative years aligned based on the starting point data.

The analysis unit 19 performs processing for performing analysis using the second observation target data. For example, the analysis unit 19 stratifies the second observation target data using the stratified data. The analysis unit 19 then displays a graph based on the stratified second observation target data on the display device. In addition, the analysis unit 19 can perform various types of processing for analysis such as causing a training model to train the relationship between the test value and/or the medical examination status of the stratified second observation target data and the risk of onset of a specific disease.

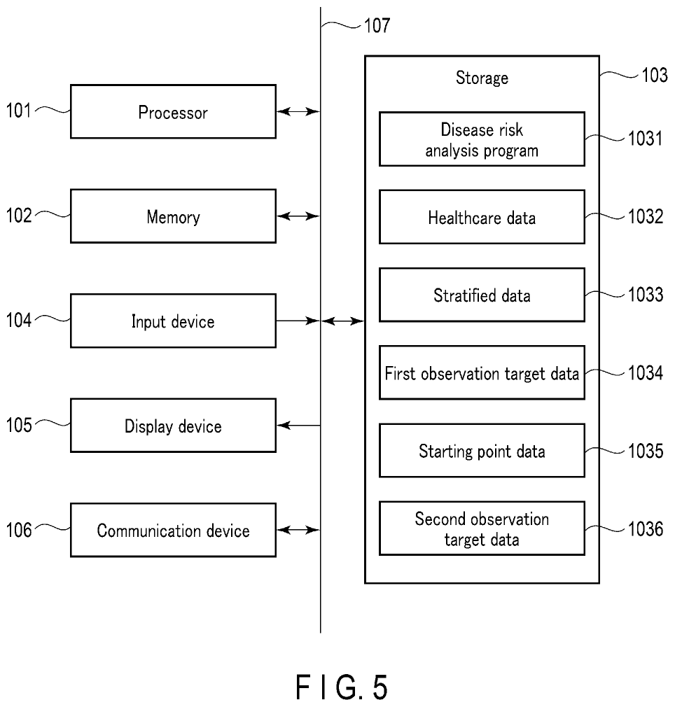

FIG. 5 is a diagram illustrating an example of a hardware configuration of the disease risk analysis apparatus 1. The disease risk analysis apparatus 1 may be a computer having a processor 101, a memory 102, a storage 103, an input device 104, a display device 105, and a communication device 106 as hardware, for example. The processor 101, the memory 102, the storage 103, the input device 104, the display device 105, and the communication device 106 are connected to a bus 107. The disease risk analysis apparatus 1 can be mounted in a terminal device such as a personal computer (PC), a smartphone, or a tablet terminal.

The processor 101 is a processor that controls the overall operation of the disease risk analysis apparatus 1. For example, the processor 101 executes a disease risk analysis program stored in the storage 103 to operate as the acquisition unit 11, the threshold determination unit 12, the stratified data generation unit 13, the criterion setting unit 14, the first observation target data generation unit 15, the starting point data generation unit 16, the second observation target data determination unit 17, the second observation target data generation unit 18, and the analysis unit 19. The processor 101 is a CPU, for example. The processor 101 may be an MPU, a GPU, an ASIC, an FPGA, or the like. The processor 101 may be a single CPU or the like, or may be a plurality of CPUs or the like.

The memory 102 includes a ROM and a RAM. The ROM is a nonvolatile memory. The ROM stores a boot program and the like for the disease risk analysis apparatus 1. The RAM is a volatile memory. The RAM is used as a work memory when, for example, the processor 101 perform processing.

The storage 103 is a storage such as a flash memory, a hard disk drive, or a solid state drive. The storage 103 stores various types of programs executed by the processor 101, such as a disease risk analysis program 1031. In addition, the storage 103 may store healthcare data 1032, stratified data 1033, first observation target data 1034, starting point data 1035, and second observation target data 1036. The healthcare data 1032, the stratified data 1033, the first observation target data 1034, the starting point data 1035, and the second observation target data 1036 are not necessarily stored in the storage 103. For example, the healthcare data 1032, the stratified data 1033, the first observation target data 1034, the starting point data 1035, and the second observation target data 1036 may be stored in a server outside of the disease risk analysis apparatus 1. In this case, the disease risk analysis apparatus 1 acquires necessary information by accessing the server using the communication device 106.

The input device 104 is an input device such as a touch panel, a keyboard, or a mouse. If the input device 104 is operated, a signal corresponding to the content of the operation is input to the processor 101 via the bus 107. The processor 101 performs various types of processing according to this signal. The input device 104 can be used to input healthcare data, determine a threshold for generating stratified data, set a criterion for determining a starting point, and determine the second observation target data, for example.

The display device 105 is a display device such as a liquid crystal display or an organic EL display. The display device 105 displays various images.

The communication device 106 is a communication device for the disease risk analysis apparatus 1 to communicate with an external apparatus. The communication device 106 may be a communication device for wired communication or a communication device for wireless communication.

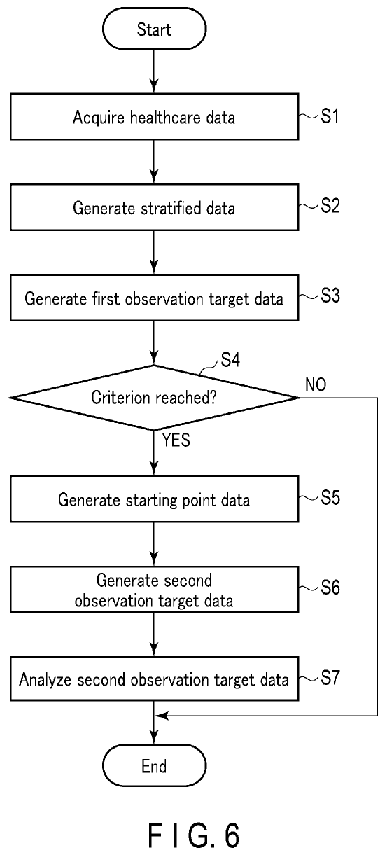

Next, the operations of the disease risk analysis apparatus 1 according to the embodiment will be described with reference to a specific example. FIG. 6 is a flowchart illustrating the operations of the disease risk analysis apparatus 1. The processing in FIG. 6 is executed by the processor 101. Prior to the following description, it is assumed that determination of a threshold by the threshold determination unit 12, setting of a criterion by the criterion setting unit 14, and determination of the second observation target data by the second observation target data determination unit 17 are performed in advance.

In step S1, the acquisition unit 11 acquires healthcare data. The healthcare data can be input to the disease risk analysis apparatus 1 in response to an operation of the disease risk analysis apparatus 1 by a disease risk analyst such as a doctor. In the following example, it is assumed that the healthcare data including the PRS data illustrated in FIG. 2, the health checkup data illustrated in FIG. 3, and the medical examination data illustrated in FIG. 4 is acquired.

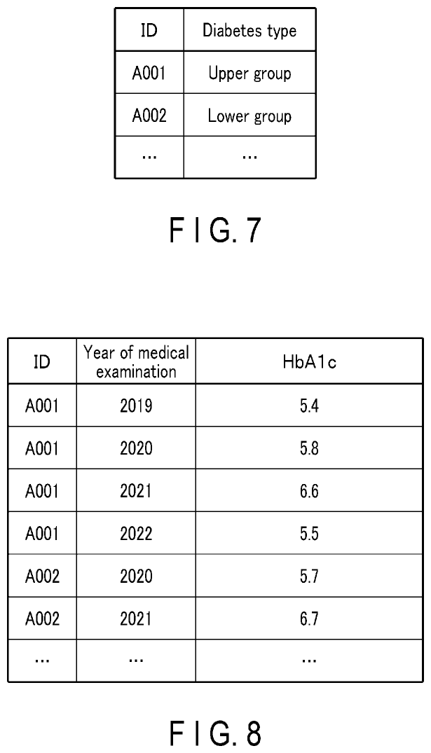

In step S2, the stratified data generation unit 13 generates stratified data by stratifying the PRS data according to the threshold determined by the threshold determination unit 12. FIG. 7 is a diagram illustrating an example of stratified data. FIG. 7 illustrates stratified data stratified by a threshold of diabetes PRS. As illustrated in FIG. 7, each ID included in the PRS data is classified into any of the “upper”, “middle”, and “lower” layers, for example, according to the result of comparison between the value of diabetes PRS and the threshold. The “upper” refers to the upper layer of diabetes PRS which is considered to be genetically susceptible to diabetes. The “lower” refers to the lower layer of diabetes PRS which is considered to be genetically less susceptible to diabetes. Although not illustrated in FIG. 7, “middle” refers to a layer not included in either “upper” or “lower”, which is considered to be moderately susceptible to diabetes.

In step S3, the first observation target data generation unit 15 generates the first observation target data from the temporal data based on the criterion set by the criterion setting unit 14. FIG. 8 is a diagram illustrating an example of first observation target data. FIG. 8 illustrates the first observation target data in a case where a criterion is set for HbA1c as an observation target. As illustrated in FIG. 8, the first observation target data in this case includes the ID, the year of medical examination, and the value of HbA1c as the test value of the observation target. The first observation target data may further include age. If a plurality of criteria such as the presence or absence of prescriptions for HbA1c and diabetes oral drug A is set, the first observation target data may include data on a plurality of test values and/or medical examination statuses.

In step S4, the starting point data generation unit 16 refers to the first observation target data and determines whether the test value and/or the medical examination status as the first observation target data have reached the criterion. For example, in a case where the observation target is HbA1c, if there is a year of medical examination in which the value of HbA1c reached a preset criterion, it is determined that the observation target has reached the criterion. In addition, in a case where the criterion is that a prescription of any of the diabetes oral drugs has been received, if there is a year of medical examination in which a prescription of any of the diabetes oral drugs was received, it is determined that the observation target has reached the criterion. In a case where a plurality of test values and/or medical examination statuses are observation targets, it may be determined that the first observation target data has reached the criterion if one piece of the first observation target data has reached the criterion, or it may be determined that the first observation target data has reached the criterion if all pieces of the first observation target data have reached the criterion. If it is determined in step S4 that the first observation target data does not reach the criterion, the process in FIG. 6 ends. If it is determined in step S4 that the first observation target data has reached the criterion, the process proceeds to step S5.

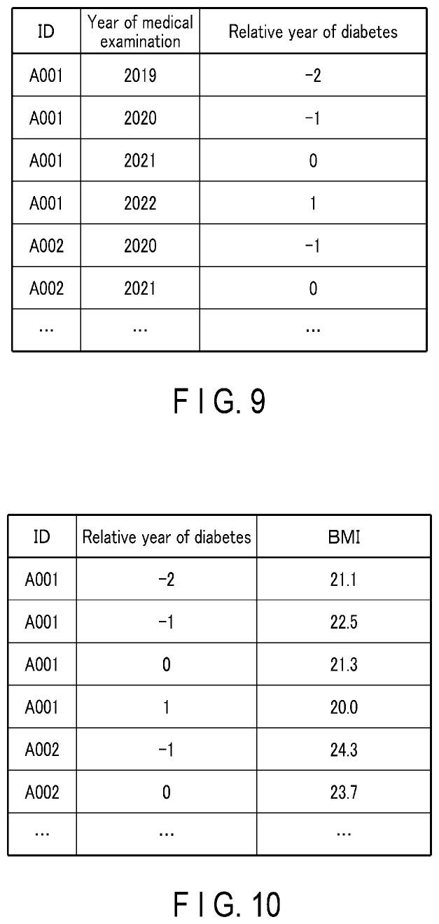

In step S5, the starting point data generation unit 16 generates starting point data from the first observation target data. FIG. 9 is a diagram illustrating an example of starting point data. The starting point data is data in which the year in which the first observation target data first reached the criterion is set as the 0th year, and the years earlier and later than the 0th year are expressed as relative years from the 0th year. That is, the starting point data includes the ID, the year of medical examination, and the relative years. For example, the user with the ID “A001” in FIG. 9 has “O” in 2021. This indicates that the value of HbA1c of the user with the ID “A001” reached the criterion in 2021 and that there was an onset of diabetes. Since the value is “0” in 2021, the value is “−2” in 2019, “−1” in 2020, and is “1” in 2022. The starting point data in FIG. 9 includes both the relative years earlier and the relative years later than the year of 2021 that is the starting point. As described above, the starting point data may include only either relative years earlier than the starting point or relative years later than the starting point.

In step S6, the second observation target data generation unit 18 generates the second observation target data based on the temporal data and the starting point data. FIG. 10 is a diagram illustrating an example of second observation target data. The second observation target data is data obtained by extracting the relative years and the observation target determined as the second observation target data in association with each other from the temporal data. FIG. 10 illustrates the second observation target data in a case where BMI is determined as an observation target. As illustrated in FIG. 10, the second observation target data in this case includes the ID, the relative year, and the value of BMI as the test value. The second observation target data may further include age. In addition, if a plurality of test values and/or a plurality of medical examination statuses is selected, the second observation target data may include data on a plurality of test values and/or medical examination statuses.

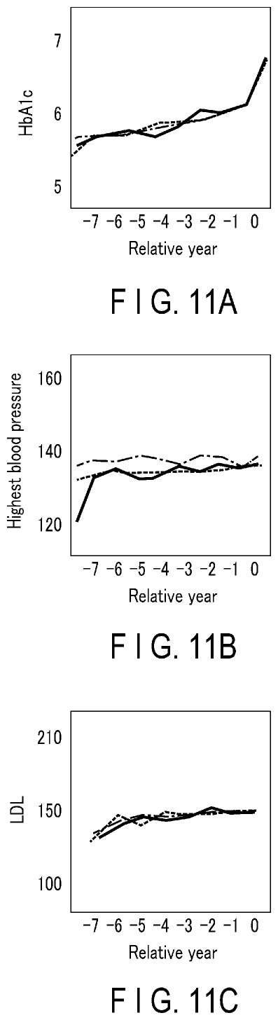

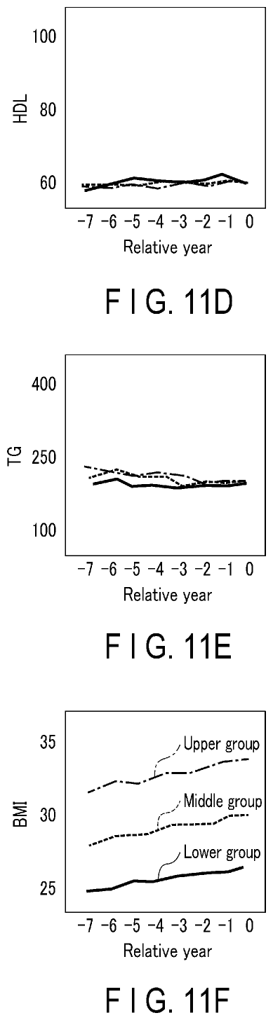

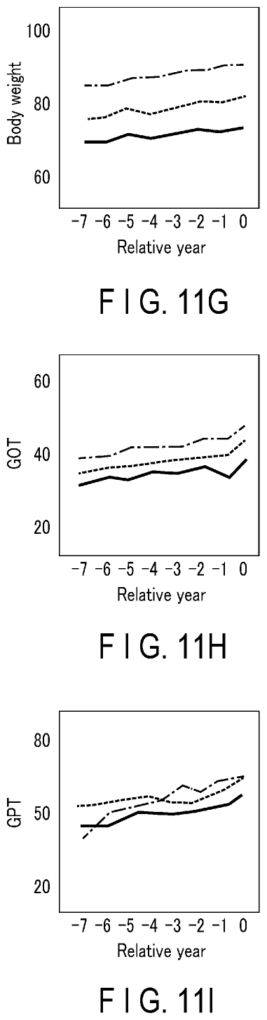

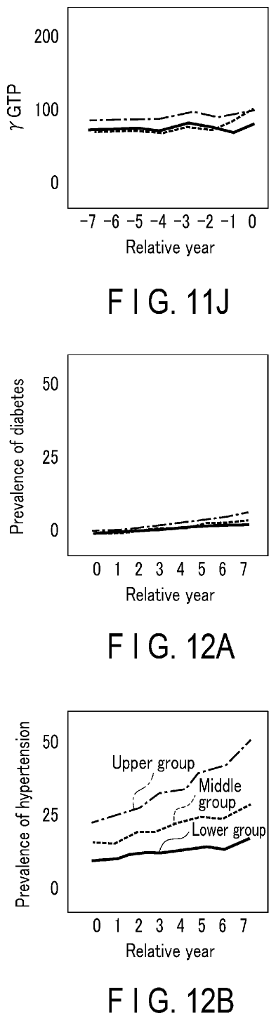

In step S7, the analysis unit 19 analyzes the second observation target data. Thereafter, the processing in FIG. 6 ends. For example, the analysis unit 19 stratifies the second observation target data by PRS using the stratified data generated in step S2. The analysis unit 19 then displays a graph of the stratified second observation target data on the display device. FIGS. 11A, 11B, 11C, 11D, 11E, 11F, 11G, 11H, 11I, and 11J are graphs obtained by selecting HbA1c, highest blood pressure, LDL, HDL, TG, BMI, body weight, GOT, GPT, and γGTP as the second observation target data, and stratifying each selected test value by the PRS of BMI. In the graphs of FIGS. 11A to 11J, the year of onset of diabetes is set as the 0th year, and the test values earlier than the 0th year are extracted as the second observation target data. The year of onset of diabetes is determined based on the fact that HbA1c as the first observation target data reached the criterion value and that diabetes oral medicine was prescribed.

The test values as the second observation target data illustrated in FIGS. 11A to 11J indicate the averages of the test values with the IDs included in the upper group, the middle group, and the lower group of the PRS of BMI. The legend in the graph illustrated in FIG. 11F can also be applied to the other graphs.

The graphs in FIGS. 11A to 11J are graphs showing the transition of test values before onset of diabetes that are stratified by the PRS of BMI. For example, with regard to the transition of BMI in FIG. 11F, there is a large difference in BMI value between the lower group and the upper group of the PRS of BMI by the year of onset of diabetes. From this, it can be seen that a person with a high PRS of BMI has a long period until diabetes is developed even if the test value of BMI is high, and a person with a low PRS of BMI has a short period until diabetes is developed even if the test value of BMI is low. As described above, it is possible to analyze whether the disease risk is different even with the same test value by aligning the years of onset of a specific disease and stratifying them by genetic differences.

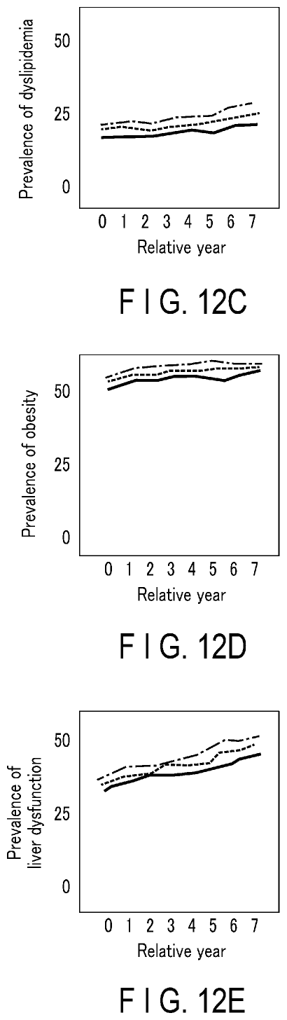

FIGS. 12A, 12B, 12C, 12D, and 12E are graphs obtained by selecting, as the second observation target data, a prevalence of diabetes, a prevalence of hypertension, a prevalence of dyslipidemia, a prevalence of obesity, and a prevalence of liver dysfunction, and stratifying each selected prevalence with the PRS of the highest blood pressure.

In the graphs of FIGS. 12A to 12E, the year in which the BMI as the first observation target data reached the criterion is set as the 0th year, the prevalence later than the 0th year is extracted as the second observation target data.

The prevalence as the second observation target data illustrated in FIGS. 12A to 12E indicates the prevalence with the IDs included in the upper group, the middle group, and the lower group of the PRS of a highest blood pressure. The legend in the graph illustrated in FIG. 12B can also be applied to the other graphs.

The graphs in FIGS. 12A, 12B, 12C, and 12E are graphs showing the transition of the prevalence of other diseases after BMI is high, that is, it is suspected that obesity has occurred, stratified by the PRS of the highest blood pressure. Generally, it is known that obesity increases the risk of hypertension, but it is found that there is a genetic difference between obesity and the onset of hypertension. As described above, it is possible to analyze whether the risk of complications after onset of a specific disease differs by aligning the years of onset of the specific disease and stratifying them by genetic differences. In FIGS. 12A to 12E, the prevalence of other complications after onset of obesity is analyzed. On the other hand, the incidence of other complications after the onset of obesity may be analyzed by the Kaplan-Meier method using each test value after the onset of obesity that is the second observation target data as an explanatory variable and using the incidence of each complication as an objective variable.

As described above, according to the present embodiment, the criterion is set for any one of the test values and/or the medical examination status of the user, and the second observation target data is generated from the temporal data and the stratified data with the year in which the test value and/or the medical examination status reached the criterion as a start point. This makes it possible to perform various analyses that cannot be performed by simple stratification using PRS.

It goes without saying that the analysis by the analysis unit 19 described above is not limited to the method of calculating the average of the test values in FIGS. 11A to 11J and the method of calculating the prevalence illustrated in FIGS. 12A to 12E. Various analyses can be performed depending on which factor is selected as each of the PRS data, the first observation target data, and the second observation target data.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A disease risk analysis apparatus comprising a processor including hardware configured to:
acquire healthcare data including genetic score data holding a genetic score for each user and temporal data including at least one of health checkup data and medical examination data for each user collected over time;
determine a threshold for stratifying the genetic score;
stratify the genetic score data based on the threshold to generate stratified data;
set a criterion for at least a test value of the health checkup data and/or at least a medical examination status of the medical examination data;
generate first observation target data by extracting a test value and/or a medical examination status corresponding to the criterion from the temporal data;
generate starting point data based on the first observation target data and the criterion;
determine an observation target from at least a test value of the health checkup data and/or at least a medical examination status of the medical examination data;
generate second observation target data by extracting the test value and/or the medical examination status determined as the observation target with the starting point data as a starting point from the temporal data; and
analyze the second observation target data by stratifying the second observation target data based on the stratified data.

2. The disease risk analysis apparatus according to claim 1, wherein
the starting point data is data of a relative year with the year in which the first observation target data reached the criterion as the starting point, and
the processor extracts, from the temporal data, a test value and/or medical examination status of a relative year earlier than the starting point, a test value and/or a medical examination status of a relative year later than the starting point, or a test value and/or a medical examination status of a relative year earlier and later than the starting point.

3. The disease risk analysis apparatus according to claim 1, wherein
the genetic score is PRS.

4. The disease risk analysis apparatus according to claim 3, wherein
the PRS includes at least one of PRS correlated with onset risk of obesity, hypertension, diabetes,
dyslipidemia, and liver dysfunction, and PRS correlated with BMI, highest blood pressure, HbA1c, LDC, HDL, TG, GOT, GPT, and γGTP.

5. The disease risk analysis apparatus according to claim 1, wherein
the processor calculates an average of the test value for each layer from the second observation target data stratified by the stratified data.

6. The disease risk analysis apparatus according to claim 1, wherein
the processor calculates a prevalence for each layer from the second observation target data stratified by the stratified data.

7. The disease risk analysis apparatus according to claim 1, wherein
the processor performs analysis by the Kaplan-Meier method with each test value in the second observation target data as an explanatory variable and an incidence of a specific disease as an objective variable.

8. The disease risk analysis apparatus according to claim 1, wherein
the first observation target data is health checkup data and/or medical examination data that serves as a criterion for determining onset of obesity, hypertension, diabetes, dyslipidemia, or liver dysfunction.

9. The disease risk analysis apparatus according to claim 1, wherein
the second observation target data is health checkup data and/or medical examination data that serves as a criterion for determining onset of obesity, hypertension, diabetes, dyslipidemia, or liver dysfunction.

10. A disease risk analysis method comprising:
acquiring healthcare data including genetic score data holding a genetic score for each user and temporal data including at least one of health checkup data and medical examination data for each user collected over time;
determining a threshold for stratifying the genetic score;
stratifying the genetic score data based on the threshold to generate stratified data;
setting a criterion for at least a test value of the health checkup data and/or at least a medical examination status of the medical examination data;
generating first observation target data by extracting a test value and/or a medical examination status corresponding to the criterion from the temporal data;
generating starting point data based on the first observation target data and the criterion;
determining an observation target from at least a test value of the health checkup data and/or at least a medical examination status of the medical examination data;
generating second observation target data by extracting the test value and/or the medical examination status determined with the starting point data as a starting point from the temporal data; and
analyzing the second observation target data by stratifying the second observation target data based on the stratified data.

11. A non-transitory processor-readable recording medium recording a disease risk analysis program for causing a processor to execute:
acquiring healthcare data including genetic score data holding a genetic score for each user and temporal data including at least one of health checkup data and medical examination data for each user collected over time;
determining a threshold for stratifying the genetic score;
stratifying the genetic score data based on the threshold to generate stratified data;
setting a criterion for at least a test value of the health checkup data and/or at least a medical examination status of the medical examination data;
generating first observation target data by extracting a test value and/or a medical examination status corresponding to the criterion from the temporal data;
generating starting point data based on the first observation target data and the criterion;
determining an observation target from at least a test value of the health checkup data and/or at least a medical examination status of the medical examination data;
generating second observation target data by extracting the test value and/or the medical examination status determined with the starting point data as a starting point from the temporal data; and
analyzing the second observation target data by stratifying the second observation target data based on the stratified data.
